(19) United States
(12) Patent Application Publication
(10) Pub. No.: US 2019/0175857 A1
(43) Pub. Date: Jun. 13, 2019

(54) BILEVEL RESPIRATORY THERAPY SYSTEM, CONTROLLER AND METHOD

(71) Applicant: Fisher & Paykel Healthcare Limited, Auckland (NZ)

(72) Inventors: David Robin WHITING, Auckland (NZ); Andrew Gordon GERRED, Auckland (NZ)

(21) Appl. No.: 16/213,770

(22) Filed: Dec. 7, 2018

Related U.S. Application Data
(60) Provisional application No. 62/596,745, filed on Dec. 8, 2017.

(51) Int. Cl. A61M 16/00; A61M 16/10

(52) U.S. Cl. CPC A61M 16/024; A61M 16/1045; A61M 2016/0015; A61M 2016/003; A61M 16/0069

(57) ABSTRACT

The present disclosure provides for non-invasive bi-level (BPAP) pressure control where the pressure of breathable gas supplied to the patient switches between an inspiration pressure during inspiration (IPAP) and another, usually lower, expiration pressure during expiration (EPAP). The present disclosure provides a system configured to prevent premature triggering to IPAP after a timed breath in spontaneous/timed mode (S/T mode) by preventing spontaneous triggering to IPAP after a timed breath until an expiration has been confirmed.

### INCORPORATION BY REFERENCE TO ANY PRIORITY APPLICATIONS

Any and all applications for which a foreign or domestic priority claim is identified in the Application Data Sheet as filed with the present application are incorporated by reference under 37 CFR 1.57 and made a part of this specification.

### FIELD OF THE DISCLOSURE

The present invention generally relates to a bi-level or non-invasive ventilation respiratory therapy system, controller and method wherein pressure of a breathable gas delivered to a patient is controlled to a higher pressure during inspiration and to a lower pressure during expiration.

### BACKGROUND

Respiratory therapy systems, controllers and methods have been proposed to treat respiratory illness, including, for example, Obstructive sleep apnea (OSA).

OSA is associated with many conditions in which there is an anatomic or functional narrowing of the patient's upper airway, and is characterized by an intermittent obstruction of the upper airway occurring during sleep. The obstruction results in a spectrum of respiratory disturbances ranging from the total absence of airflow (apnea) to significant obstruction with or without reduced airflow (hypopnea and snoring), despite continued respiratory efforts. The morbidity of the syndrome arises from hypoxemia, hypercapnia, bradycardia and sleep disruption associated with the apneas and subsequent arousals from sleep.

Positive airway pressure (PAP) therapy has become the mainstay of treatment in Obstructive Sleep Disordered Breathing (OSDB), which includes Obstructive Sleep Apnea, Upper Airway Resistance Syndrome, Snoring, exaggerations of sleep-induced increases in the collapsibility of the upper airway and all conditions in which inappropriate collapsing of a segment of the upper airway causes significant un-physiologic obstruction to airflow. This collapse generally occurs whenever pressure in the collapsible portion of the airway decreases below a level defined as a “critical tissue pressure” in the surrounding wall. The PAP therapy is directed to maintaining pressure in the collapsible portion of the airway at or above the critical tissue pressure at all times. It is well known during PAP therapy to increase the pressure delivered to the patient's airway to a level higher than this critical tissue pressure at all times when the patient is wearing the device. The applied pressure is either a constant pressure, or a pressure based on breath-by-breath determination of the need for treatment.

Non-invasive ventilation (NIV) is used to ventilate a patient without requiring intubation. In NIV, the patient interface does not enter the person's body, or minimally enters the body, and no unnatural channels are required to gain access to the airway. NIV is used to breathe for the patient, or can be used to help the breathing of a patient, in which case the patient's spontaneous breathing effort triggers the respiratory apparatus to deliver the appropriate pressure. Generally, the patient partially exhales through exhaust ports in the mask and exhales the balance back into the mask and gas delivery tubing. The positive pressure being applied from the respiratory apparatus opens the upper airway, using a patient interface mask that generally seals over the nose and or mouth, or seals inside the nose.

### SUMMARY

In accordance with certain features, aspects and advantages of at least one of the embodiments disclosed herein, a respiratory apparatus that provides a flow of gases to a patient at different pressures during inspiratory and expiratory cycles of breathing, the respiratory apparatus comprising: a controller configured to control the pressure of breathable gas delivered to the patient by a flow generator and to receive one or more signals from one or more sensors indicative of the pressure and/or flow of the gas in the system, wherein the controller is configured to: control the flow generator to deliver breathable gas at an exhalation pressure (EPAP) after delivering breathable gas at an inhalation pressure (IPAP) for a defined duration; detect inspiration based on when flow rate data indicates that the flow rate is above a trigger threshold and an exhalation check indicator indicates that the patient has previously exhaled; and in response to detection of inspiration, deliver breathable gas at the IPAP.

In some configurations, the defined duration is a machine breath duration.

In some configurations, after the machine breath duration, the controller transitions to EPAP regardless of the flow rate.

In some configurations, the defined duration is a maximum duration at IPAP.

In some configurations, the controller is configured to detect exhalation based at least in part on whether the flow rate data indicates that the flow rate is below a cycling threshold.

In some configurations, the trigger threshold and the cycling threshold have different flow rate threshold values.

In some configurations, on transition to IPAP, the controller is configured to change the expiration check indicator to indicate that exhalation has not occurred.

In some configurations, the expiration check indicator is a Boolean operator.

In some configurations, the controller is configured to deliver breathable gas at the IPAP if inspiration is not detected within a backup time period.

In some configurations, the trigger threshold varies during the inspiratory and/or expiratory cycles of breathing.

In accordance with certain features, aspects and advantages of at least one of the embodiments disclosed herein, a method for controlling a respiratory apparatus that provides a flow of gases to a patient at different pressures during inspiratory and expiratory cycles of breathing, the method comprising: controlling the pressure of breathable gas delivered to the patient by a flow generator; receiving one or more signals from one or more sensors indicative of the pressure and/or flow of the gas in the system; controlling the flow generator to deliver breathable gas at an exhalation pressure (EPAP) after delivering breathable gas at an inhalation pressure (IPAP) mode for a defined duration; detecting inspiration based on when flow rate data indicates that the flow rate is above a trigger threshold and an exhalation check indicator indicates that the patient has previously exhaled; and in response to detection of inspiration, delivering breathable gas in the IPAP.

In some configurations, the defined duration is a machine breath duration.

In some configurations, after the machine breath duration, the controller transitions to EPAP regardless of the flow rate.

In some configurations, the defined duration is a maximum duration at the IPAP.

In some configurations, the method comprises detecting exhalation based at least in part on whether the flow rate data indicates that the flow rate is below a cycling threshold.

In some configurations, the trigger threshold and the cycling threshold have different flow rate threshold values.

In some configurations, the method comprises on transition to the IPAP, changing the expiration check indicator to indicate that exhalation has not occurred.

In some configurations, the expiration check indicator is a Boolean operator

In some configurations, the method comprises delivering breathable gas in the IPAP if inspiration is not detected within a backup time period.

In some configurations, the trigger threshold varies during the inspiratory and/or expiratory cycles of breathing.

Features from one or more embodiments or configurations may be combined with features of one or more other embodiments or configurations. Additionally, more than one embodiment may be used together during a process of respiratory support of a patient.

The term ‘comprising’ as used in this specification means ‘consisting at least in part of’. When interpreting each statement in this specification that includes the term ‘comprising’, features other than that or those prefaced by the term may also be present. Related terms such as ‘comprise’ and ‘comprises’ are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

It should be understood that alternative embodiments or configurations may comprise any or all combinations of two or more of the parts, elements or features illustrated, described or referred to in this specification.

This invention may also be said broadly to consist in the parts, elements and features referred to or indicated in the specification of the application, individually or collectively, and any or all combinations of any two or more said parts, elements or features.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting. Where specific integers are mentioned herein which have known equivalents in the art to which this invention relates, such known equivalents are deemed to be incorporated herein as if individually set forth.

### BRIEF DESCRIPTION OF THE DRAWINGS

FIG. 1 shows in diagrammatic form a flow therapy apparatus.

FIG. 2A illustrates a graph of a flow therapy session without an expiratory check control system.

FIG. 2B illustrates a graph of a flow therapy session with an expiratory check control system.

FIG. 3 illustrates a graph of a flow therapy session with an expiratory check control system.

FIG. 4 illustrates a process for controlling operation of a flow therapy apparatus

### DETAILED DESCRIPTION

### Flow Therapy Apparatus

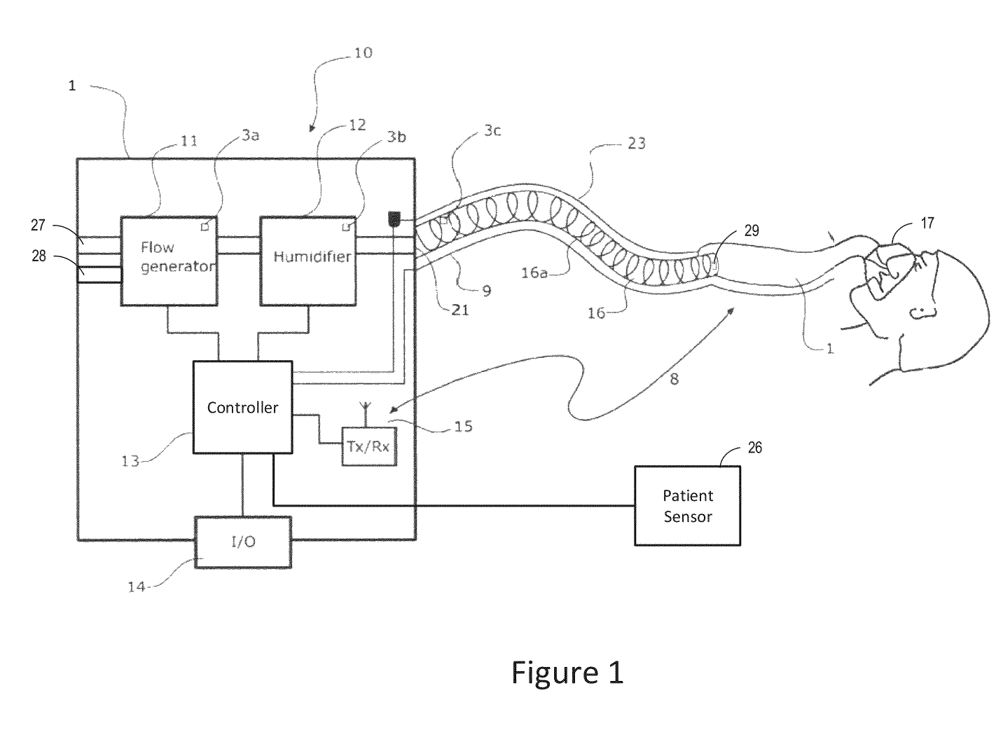

A flow therapy apparatus 10 is shown in FIG. 1. The apparatus 10 can comprise a main housing 1 that contains a flow generator 11 in the form of a motor/impeller arrangement (for example, a blower), an optional humidifier 12, a controller 13, and a user interface 14 (comprising, for example, a display and input device(s) such as button(s), a touch screen, or the like). The controller 13 can be configured or programmed to control the operation of the apparatus. For example, the controller can control components of the apparatus, including but not limited to: operating the flow generator 11 to create a flow of gas (gases flow) for delivery to a patient, operating the humidifier 12 (if present) to humidify and/or heat the generated gases flow, optionally control a flow of oxygen into the flow generator blower, receiving user input from the user interface 14 for reconfiguration and/or user-defined operation of the apparatus 10, and outputting information (for example on the display) to the user. The user can be a patient, healthcare professional, or anyone else interested in using the apparatus. As used herein, a “gases flow” can refer to any flow of gases that may be used in the breathing assistance or respiratory device, such as a flow of ambient air, a flow comprising substantially 100% oxygen, a flow comprising some combination of ambient air and oxygen, and/or the like. The gases flow may further include flow from a nebulizer.

A patient breathing conduit 16 is coupled at one end to a gases flow outlet 21 in the housing 1 of the flow therapy apparatus 10. The patient breathing conduit 16 is coupled at another end to a patient interface 17 such as face mask. Additionally, or alternatively, the patient breathing conduit 16 can be coupled to a non-sealed nasal cannula with a manifold and nasal prongs, a nasal mask, a nasal pillows mask, an endotracheal tube, a tracheostomy interface, and/or the like. The gases flow that is generated by the flow therapy apparatus 10 may be humidified, and delivered to the patient via the patient conduit 16 through the patient interface 17. The patient conduit 16 can have a heater wire 16a to heat gases flow passing through to the patient. The heater wire 16a can be under the control of the controller 13. The patient conduit 16 and/or patient interface 17 can be considered part of the flow therapy apparatus 10, or alternatively peripheral to it. The flow therapy apparatus 10, breathing conduit 16, and patient interface 17 together can form a flow therapy system.

The controller 13 can control the flow generator 11 to generate a gases flow of the desired flow rate. The controller 13 can also control a supplemental oxygen inlet to allow for delivery of supplemental oxygen, the humidifier 12 (if present) can humidify the gases flow and/or heat the gases flow to an appropriate level, and/or the like. The gases flow is directed out through the patient conduit 16 and cannula 17 to the patient. The controller 13 can also control a heating element in the humidifier 12 and/or the heating element 16a in the patient conduit 16 to heat the gas to a desired temperature for a desired level of therapy and/or level of comfort for the patient. The controller 13 can be programmed with or can determine a suitable target temperature of the gases flow.

The oxygen inlet port 28 can include a valve through which a pressurized gas may enter the flow generator or blower. The valve can control a flow of oxygen into the flow generator blower. The valve can be any type of valve, including a proportional valve or a binary valve. The source of oxygen can be an oxygen tank or a hospital oxygen supply. Medical grade oxygen is typically between 95% and 100% purity. Oxygen sources of lower purity can also be used. Examples of valve modules and filters are disclosed in U.S. Provisional Application No. 62/409,543, titled “Valve Modules and Filter”, filed on Oct. 18, 2016, and U.S. Provisional Application No. 62/488,841, titled “Valve Modules and Filter”, filed on Apr. 23, 2017, which are hereby incorporated by reference in their entireties.

The flow therapy apparatus 10 can measure and control the oxygen content of the gas being delivered to the patient, and therefore the oxygen content of the gas inspired by the patient.

Operation sensors 3a, 3b, 3c, such as flow, temperature, humidity, and/or pressure sensors can be placed in various locations in the flow therapy apparatus 10. Additional sensors may be placed in various locations on the patient conduit and/or face mask (for example, there may be a temperature sensor 29 at or near the end of the inspiratory tube). Output from the sensors can be received by the controller 13, to assist the controller in operating the flow therapy apparatus 10 in a manner that provides suitable therapy. In some configurations, providing suitable therapy includes meeting a patient's peak inspiratory demand. The apparatus 10 may have a transmitter and/or receiver 15 to enable the controller 13 to receive signals 8 from the sensors and/or to control the various components of the flow therapy apparatus 10, including but not limited to the flow generator 11, humidifier 12, and heater wire 16a, or accessories or peripherals associated with the flow therapy apparatus 10. Additionally, or alternatively, the transmitter and/or receiver 15 may deliver data to a remote server or enable remote control of the apparatus 10.

Oxygen may be measured by placing one or more gas composition sensors (such as an ultrasound transducer system) after the oxygen and ambient air have finished mixing. The measurement can be taken within the device, the delivery conduit, the patient interface, or at any other suitable location.

Oxygen concentration may also be measured by using flow rate sensors on at least two of the ambient air inlet conduit, the oxygen inlet conduit, and the final delivery conduit to determine the flow rate of at least two gases. By determining the flow rate of both inlet gases or one inlet gas and one total flow rate, along with the assumed or measured oxygen concentrations of the inlet gases (about 20.9% for ambient air, about 100% for pure oxygen), the oxygen concentration of the final gas composition can be calculated. Alternatively, flow rate sensors can be placed at all three of the ambient air inlet conduit, the oxygen inlet conduit, and the final delivery conduit to allow for redundancy and testing that each sensor is working correctly by checking for consistency of readings. Other methods of measuring the oxygen concentration delivered by the flow therapy apparatus 10 can also be used.

The flow therapy apparatus 10 can include a patient sensor 26, such as a pulse oximeter, to measure one or more physiological parameters of the patient, such as a patient's blood oxygen saturation (SpO₂), heart rate, respiratory rate, perfusion index, and provide a measure of signal quality. The sensor 26 can communicate with the controller 13 through a wired connection or by communication through a wireless transmitter on the sensor 26. The sensor 26 may be a disposable adhesive sensor designed to be connected to a patient's finger. The sensor 26 may be a non-disposable sensor. Sensors are available that are designed for different age groups and to be connected to different locations on the patient, which can be used with the flow therapy apparatus. The pulse oximeter would be attached to the user, typically at their finger, although other places such as an earlobe are also an option. The pulse oximeter would be connected to a processor in the device and would constantly provide signals indicative of the patient's blood oxygen saturation.

The flow generator or blower 11 can include an ambient air inlet port 27 to entrain ambient room air into the blower. The flow therapy apparatus 10 may also include an oxygen inlet port 28 leading to a valve through which a pressurized gas may enter the flow generator or blower 11. The valve can control a flow of oxygen into the flow generator blower 11. The valve can be any type of valve, including a proportional valve or a binary valve.

The blower can operate at a motor speed of greater than about 1,000 RPM and less than about 50,000 RPM, greater than about 2,000 RPM and less than about 26,000 RPM, or between any of the foregoing values. Operation of the blower can mix the gases entering the blower through the inlet ports. Using the blower as the mixer can decrease the pressure drop that would otherwise occur in a system with a separate mixer, such as a static mixer comprising baffles, because mixing requires energy.

### IPAP/EPAP Control

The present disclosure provides for non-invasive bi-level (BPAP) pressure control where the pressure of breathable gas supplied to the patient switches between an inspiration pressure during inspiration (IPAP) and another, usually lower, expiration pressure during expiration (EPAP). Bi-level PAP control is known in the art and is described in U.S. Pat. Nos. 5,148,802, 5,433,193 and 5,134,995 the entire contents of each of which are incorporated herein by reference. The present disclosure provides a system configured to prevent premature triggering to IPAP after a timed breath in spontaneous/timed mode (S/T mode) by preventing spontaneous triggering to IPAP after a timed breath until an expiration has been confirmed.

A bi-level respiratory therapy system can detect the onset of inspiration and expiration from data received from the one or more pressure and/or flow sensors in the system. For example the sensors may be in the gas delivery conduit, the patient interface, and/or one or more connectors of the gas delivery conduit. During inspiration, the system increases the pressure of the breathable gas delivered to the patient to the inspiration pressure (IPAP). During expiration, the system reduces the pressure of the breathable gas delivered to the patient to the, lower, expiration pressure (EPAP). The expiration pressure may be predetermined to be a fixed amount less than the delivered inspiration pressure and may therefore be considered to be expiratory pressure relief. The system may therefore be configured to automatically drop the pressure of the delivered gas by, for example, 2-4 cm H₂O, once expiration is detected. A typical pressure difference between IPAP and EPAP when treating OSA would be about 3 cm H₂O, for example. Any other suitable pressure difference between IPAP and EPAP may be selected as required. The inspiration pressure may be set in advance by the clinician, or by the clinician during set-up/titration of the system, or by the system controller in accordance with one or more predetermined algorithms and/or calculations.

In bi-level therapy, pressure can be provided to a patient at the inspiratory pressure (IPAP) and the expiratory pressure (EPAP) that is generally timed or synchronized with the patient's spontaneous breathing and with or without a backup rate. The modes of operation generally include spontaneous mode, spontaneous/timed mode, and timed mode.

In spontaneous mode (S-mode), the flow generator delivers breathable gas at an inspiration pressure (IPAP) on detection of inspiration, and at a lower expiratory pressure (EPAP) on detection of expiration. Triggering the change in pressure from EPAP to IPAP is based on the detection of patient inspiration. Typically, there is a maximum duration for which IPAP can last (Dₘₐₓ), so the system will not stay at IPAP in the event of a leak.

In spontaneous/timed mode (S/T mode), patient breathing will trigger the change between EPAP and IPAP. Additionally, a backup breath, also referred to as a timed breath or machine breath, may be triggered by the system if inspiration is not detected within a backup duration or in accordance with a backup rate. In a preferred embodiment, the backup duration is a set time defined from the previous trigger to inspiration, the previous trigger to IPAP being either a spontaneously-triggered breath or a machine-triggered breath. The backup duration and/or the backup rate can be set by a clinician. The backup breath will typically have a set duration (Dᵢ); that is, if the backup breath is triggered, IPAP will be applied for Dᵢ, regardless of the patient's spontaneous breathing. Alternatively, IPAP may be applied for Dᵢ for all breaths, regardless of whether they are triggered by the patient's spontaneous breathing or by reaching the backup time or rate.

In timed mode, IPAP and EPAP are provided at a timing set by a clinician, regardless of patient's spontaneous breathing.

The system may include a backup measurement being a backup time related variable such as a backup time period/duration or backup frequency/rate associated with the mode of therapy. The backup time related variable is a clinician set time period which is set to try to correspond to the patient's breathing rate/breath duration. The system controller can have a timer which compares the set backup time related variable with corresponding time-related characteristic of the breath, such as for example, the actual duration of a breath. If a breath is not detected in accordance with the backup time related variable, for example within the backup duration, the system automatically initiates a new breath by switching to the higher IPAP pressure. Effectively, the system times the patient's breathing rate and/or duration, and in particular the rate and/or duration of at least the expiratory portion of the breath and if the patient takes too long to take a breath, the system switches to IPAP to try to force the patient to start spontaneously breathing again.

During S/T mode, when a patient is breathing spontaneously with a breathing rate quicker than the set backup rate (i.e. breath duration shorter than the “backup duration”) the system will oscillate between a clinician set IPAP and EPAP during inhalation and exhalation, respectively. In this situation the backup time related variable control methodology is not triggered.

If a single breath duration exceeds the backup duration, the system will automatically try to initiate a backup breath by transitioning to the clinician-set IPAP. This is intended to provide a guarantee that the system switches between IPAP and EPAP at a rate no less than the backup rate intended by the clinician. For example, if the patient takes a breath and inhales every 5 seconds, the backup duration may be set at 6 seconds from the start of inspiration. If the backup time related variable measurement is a backup time period, the expiratory portion of the patient's breathing may be observed, measured or determined, to last on average 3 seconds. The backup duration may then be set at 4 seconds from the start of expiration. The above example has been provided in terms of time duration, typically measured in seconds, but could alternatively be provided in terms of rate or frequency, typically measured as breaths per minute (BPM).

IPAP, EPAP and the backup time related variable may be set by a clinician during set-up of the system with a given patient, or may be determined automatically by the system. The system may incorporate control methodology to automatically adjust the IPAP, EPAP and/or backup time related variable in response to signals received from one or more pressure and/or flow sensors and/or other sensors in the system.

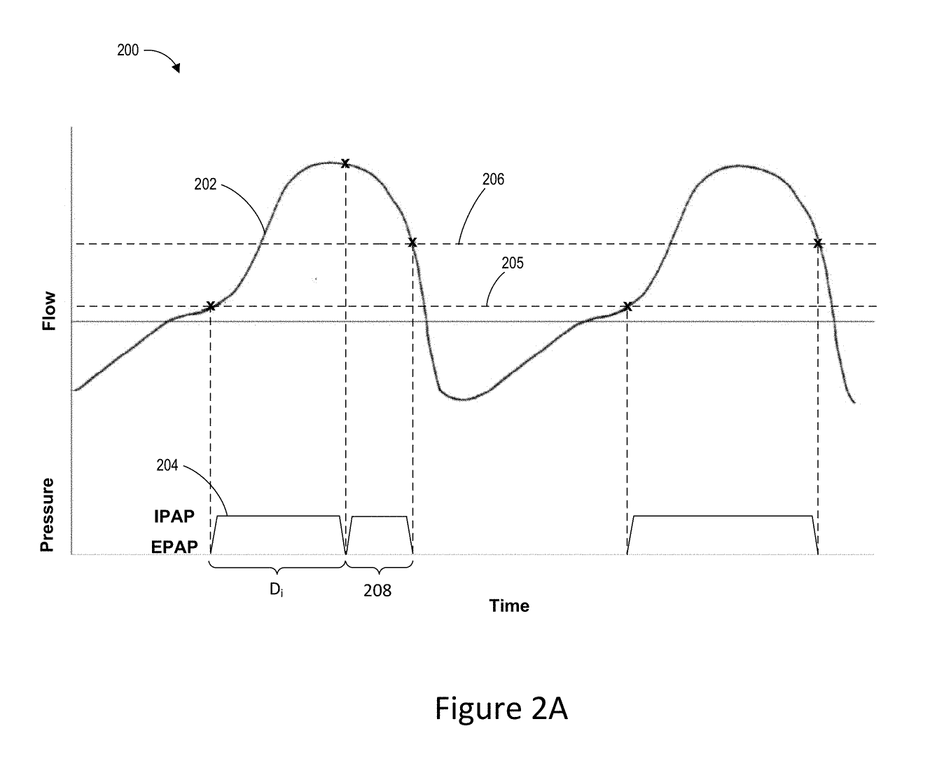

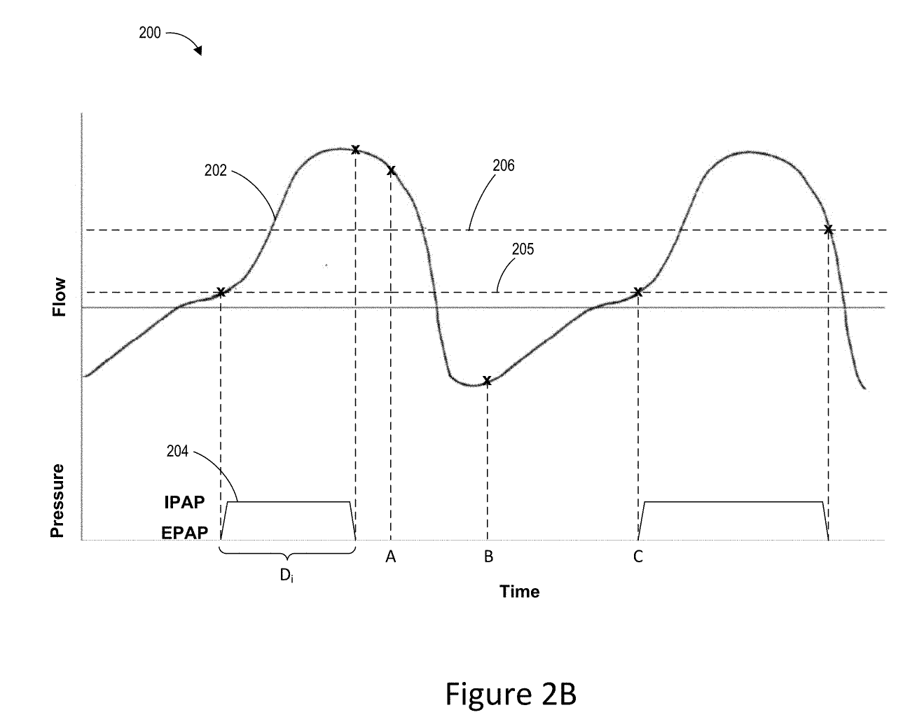

FIG. 2A illustrates an example of a graph 200 illustrating respiratory data for a patient where an expiratory check is not used. The graph 200 provides a flow rate curve over time 202 and a pressure curve over time 204. The pressure curve oscillates between IPAP and EPAP. If EPAP is being provided and a flow rate is above the IPAP trigger threshold 205 (also referred to as a trigger threshold), the device will then change to provide IPAP. If IPAP is being provided and the flow rate is below an EPAP cycling threshold 206 (also referred to as a cycling threshold), the device will then change to provide EPAP. In some configuration, the trigger threshold 205 and cycling threshold 206 can be constant values, such as illustrated by dashed lines 205 and 206 in FIGS. 2A and 2B. The trigger threshold 205 and the cycling threshold 206 used to determine inspiration and expiration of a patient may be based on flow rates.

In S/T mode, if a patient has not initiated a breath after a defined period of time, a machine breath, or backup breath, is provided by supplying an IPAP pressure for a set period of time, Dᵢ, after which the pressure will cycle to EPAP. Dᵢ may be set to an expected inspiration duration by a clinician. This cycling can occur regardless of whether the patient is still inspiring.

Alternatively, a minimum and maximum IPAP duration may be provided. This may be for just machine breaths, just for spontaneously triggered breaths, or for all breaths regardless of whether the breath was spontaneously triggered or provided as a machine breath. IPAP will be provided for at least the minimum IPAP duration, regardless of whether the flow rate has dropped below the cycling threshold, and will cycle to EPAP at the maximum IPAP duration, regardless of whether the flow rate is still above the cycling threshold.

On occasion the patient's inspiration may be longer than Dᵢ or the maximum IPAP duration, such as illustrated in FIG. 2A. For example, the machine breath may trigger the patient to take a longer or deeper breath than is typical. In this scenario, after the Dᵢ has ended and the pressure dropped to EPAP, the detected flow rate is still above the triggering threshold, in which case the device immediately triggers back to IPAP after the machine breath, causing an uncomfortable dip and rise in pressure illustrated by section 208.

FIG. 2B illustrates an example of the graph 200 illustrating respiratory data for a patient where an expiratory check is used. In order to prevent the unexpected triggering of IPAP, the present disclosure provides that following a machine breath or after the maximum IPAP duration has been exceeded, a change to IPAP for a spontaneously triggered breath may not occur until after an expiration has been detected, the expiration occurring since the previous transition to IPAP. As such, the system can be configured so that a spontaneous change to IPAP can only be triggered by the patient's breathing when two conditions are satisfied: 1) the flow rate is greater than the IPAP triggering threshold and 2) when expiration has been detected since the previous trigger to IPAP.

The expiration check can be an indicator that can indicate whether expiration has been detected since the inhalation. For example, the expiration check can be a flag that is a Boolean operator that can be changed from false to true after expiration has been detected. The expiration check indicator can be reset at the beginning of each inspiration. The system can detect expiration by detecting the transition from inspiration to expiration, by detecting whether the flow rate is below the cycling threshold 206, or by using other methods of detecting expiration that are known in the art. A machine breath may still be applied if the backup rate is reached and the flow rate has not been detected above the triggering threshold, regardless of the expiration check.

With further reference to FIG. 2B, the graph 200 provides a flow rate curve over time 202 and a pressure curve over time 204. Points A, B, and C provide examples of application of the expiratory check. At A, the pressure is at EPAP, the flow is greater than the cycling threshold 206 and the expiration check indicator is false. Accordingly, the system does not trigger to IPAP. At B, the flow is less than the trigger threshold 205 and cycling threshold 206, and the expiration check indicator is true. Accordingly, the system does not trigger to IPAP. At C, the flow satisfies the trigger threshold 205 and the expiration check indicator is true. Accordingly, the system does trigger to IPAP.

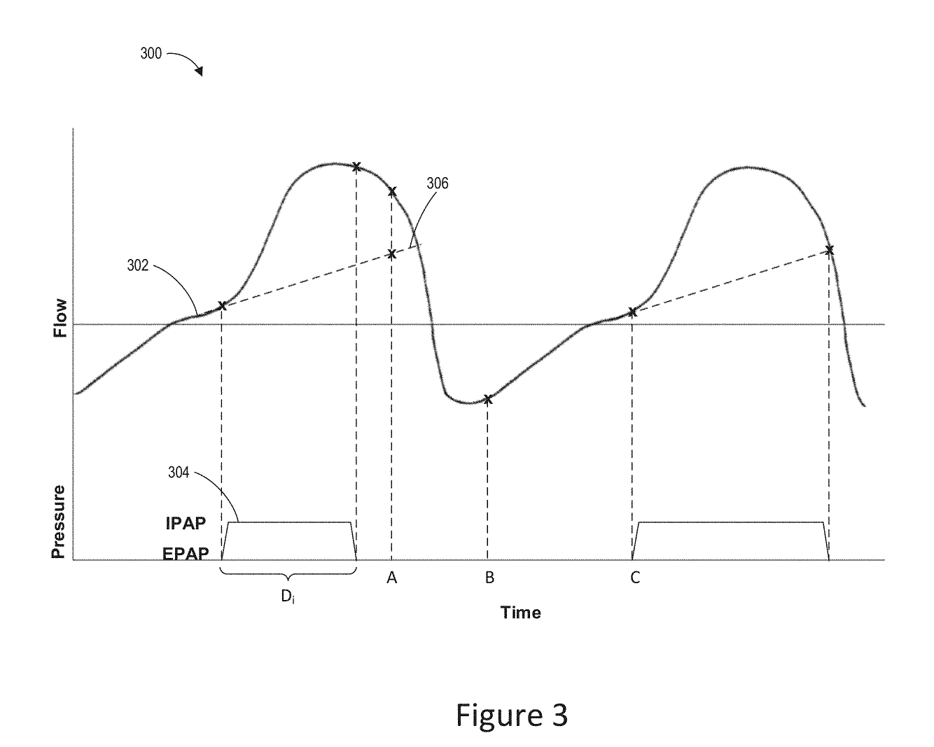

FIG. 3 illustrates another embodiment of a flow threshold 306 that changes over time. In the illustrated configuration, the trigger threshold and/or the cycling threshold do not have constant values, rather a flow threshold 306 changes during inspiration and/or expiration. For example, after transition to IPAP in each breath, the threshold 306 can change (e.g., increase) during inspiration. A temporary offset can be added to the estimated leak flow rate reference signal. The offset can be proportional to the integral of estimated patient flow rate beginning at initiation of the inspiratory breath so that it gradually increases with time during inspiration at a rate proportional to the patient's inspiratory flow rate. In this way, the EPAP cycling threshold comes up to meet the inspiratory flow rate.

The graph 300 provides a flow rate curve over time 302 and a pressure curve over time 304. Points A, B, and C provide examples of application of the expiratory check. At A, the pressure is at EPAP, the flow is greater than the threshold 306 and the expiration check indicator is false. Accordingly, the system does not trigger to IPAP. At B, the flow is less than the threshold 306, and the expiration check indicator is true. Accordingly, the system does not trigger to IPAP. At C, the flow satisfies the threshold 306 and the expiration check indicator is true. Accordingly, the system does trigger to IPAP

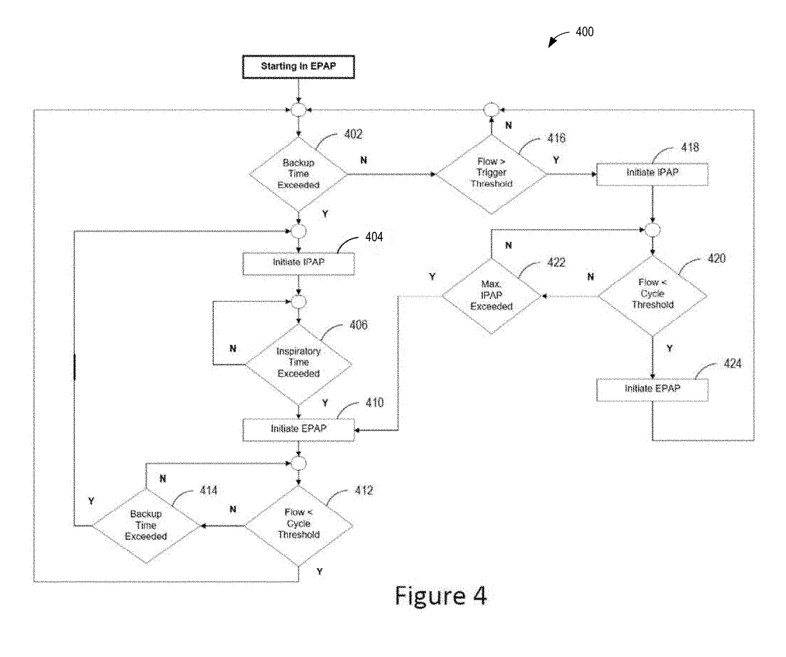

FIG. 4 provides a flowchart for a process 400 for a flow therapy apparatus operating in a spontaneous/timed mode during a therapy session. The process 400 can be implemented at least in part by the controller 13, the flow generator, inlet valves 27, 28, and/or other components of the system.

The process starts in EPAP. While the system is at EPAP, it receives data associated with the respiratory session. After the system has been powered on, it may have a warm up period prior to initiating the therapy session and entering bi-level or NIV mode. The data can be received from the one or more operational sensors, such as pressure sensors, flow sensors, timers, and/or other sensors, and/or other components of the flow therapy apparatus. The controller can analyse the data to control the mode of operation of flow therapy apparatus. Additionally, the system can analyse the received data to determine whether to update one or more parameters. For example, the expiration check indicator can be updated based on a determination that an expiration occurred (e.g., change the indicator from false to true).

At block 402, the controller determines whether the backup time threshold has been exceeded. For example, the controller determines whether a threshold for a backup duration and/or backup rate has been exceeded since the previous inspiration. If the backup time threshold has not been exceeded the process proceeds to block 416. If the backup time threshold has been exceeded the process proceeds to block 404.

At block 404, the controller initiates a machine breath, increasing the pressure to the IPAP. The machine breath can be for a fixed duration, Dᵢ, as described above. After the duration of the machine breath, the controller transitions to block 406. At block 406, the controller determines whether the machine breath duration, Dᵢ, has been exceeded. If the machine breath duration has been exceeded, the process proceeds to block 410. If the time has not been exceeded the process returns to block 406 to determine if the inspiratory time has been exceeded.

At block 410, the controller initiates EPAP. At block 412, the controller determines whether the flow rate is less than an EPAP cycling threshold. The cycling threshold can be a constant value, such as illustrated by the cycling threshold 206 in FIGS. 2A and 2B, or a threshold that changes during inspiration, such as illustrated by the threshold 306 illustrated in FIG. 3. If the flow rate is less than the cycling threshold, the process transitions to block 402, and the process restarts. If not, the process proceeds to block 414. At block 414, the controller determines whether the backup time for EPAP has been exceeded. If the time has been exceeded, the process transitions to block 404 to initiate IPAP again. Otherwise process returns to block 412. The expiration check indicator can be reset when the process proceeds to block 402.

At block 416, after determining that the backup time has not been exceeded, the controller determines whether the flow rate is greater than the IPAP trigger threshold (such as trigger threshold 205). If not, the controller returns to block 402. If the flow rate is greater than the trigger threshold, the process proceeds to block 418. At block the 418, the controller initiates IPAP. The IPAP may extend for at least a minimum duration but not longer than a maximum duration, as described above. At block 420, during IPAP, the controller determines when the flow rate drops below the cycling threshold (such as cycling threshold 206). If the flow rates drops below the cycling threshold, the process proceeds to block 424 to initiate EPAP and returns to block 402 to start the process over. If the flow is not less than the cycling threshold the process proceeds to block 422. At block 422, the controller determines whether the maximum inspiration time threshold has been exceeded. If the maximum inspiration time threshold has been exceeded, the process proceeds to block 410 to initiate EPAP and proceeds with the process as described above. If the maximum inspiration time threshold has not been exceeded, the process returns to block 420.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The terms “approximately,” “about,” and “substantially” as used herein represent an amount close to the stated amount that still performs a desired function or achieves a desired result. For example, in some embodiments, as the context may permit, the terms “approximately”, “about”, and “substantially” may refer to an amount that is within less than or equal to 10% of, within less than or equal to 5% of, and within less than or equal to 1% of the stated amount.

Reference to any prior art in this specification is not, and should not be taken as, an acknowledgement or any form of suggestion that that prior art forms part of the common general knowledge in the field of endeavour in any country in the world.

The disclosed apparatus and systems may also be said broadly to consist in the parts, elements and features referred to or indicated in the specification of the application, individually or collectively, in any or all combinations of two or more of said parts, elements or features.

Where, in the foregoing description reference has been made to integers or components having known equivalents thereof, those integers are herein incorporated as if individually set forth.

Depending on the embodiment, certain acts, events, or functions of any of the algorithms, methods, or processes described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially.

It should be noted that various changes and modifications to the presently preferred embodiments described herein will be apparent to those skilled in the art. Such changes and modifications may be made without departing from the spirit and scope of the disclosed apparatus and systems and without diminishing its attendant advantages. For instance, various components may be repositioned as desired. It is therefore intended that such changes and modifications be included within the scope of the disclosed apparatus and systems. Moreover, not all of the features, aspects and advantages are necessarily required to practice the disclosed apparatus and systems. Accordingly, the scope of the disclosed apparatus and systems is intended to be defined only by the claims that follow.

1. A respiratory apparatus that provides a flow of gases to a patient at different pressures during inspiratory and expiratory cycles of breathing, the respiratory apparatus comprising:
a controller configured to control the pressure of breathable gas delivered to the patient by a flow generator and to receive one or more signals from one or more sensors indicative of the pressure and/or flow of the gas in the system, wherein the controller is configured to:
control the flow generator to deliver breathable gas at an exhalation pressure (EPAP) after delivering breathable gas at an inhalation pressure (IPAP) for a defined duration;
detect inspiration based on when flow rate data indicates that the flow rate is above an trigger threshold and an exhalation check indicator indicates that the patient has previously exhaled; and
in response to detection of inspiration, deliver breathable gas at the IPAP.

2. The respiratory apparatus of claim 1, wherein the defined duration is a machine breath duration.

3. The respiratory apparatus of claim 2, wherein after the machine breath duration, the controller transitions to EPAP regardless of the flow rate.

4. The respiratory apparatus of claim 1, wherein the defined duration is a maximum duration in the IPAP.

5. The respiratory apparatus of claim 1, wherein the controller is configured to detect exhalation based at least in part on whether the flow rate data indicates that the flow rate is below a cycling threshold.

6. The respiratory apparatus of claim 5, wherein the trigger threshold and the cycling threshold have different flow rate threshold values.

7. The respiratory apparatus of claim 1, wherein, on transition to IPAP, the controller is configured to change the expiration check indicator to indicate that exhalation has not occurred.

8. The respiratory apparatus of claim 1, wherein the expiration check indicator is a Boolean operator

9. The respiratory apparatus of claim 1, wherein the controller is configured to deliver breathable gas at the IPAP if inspiration is not detected within a backup time period.

10. The respiratory apparatus of claim 1, wherein the trigger threshold varies during the inspiratory and/or expiratory cycles of breathing.

11. A method for controlling a respiratory apparatus that provides a flow of gases to a patient at different pressures during inspiratory and expiratory cycles of breathing, the method comprising:
controlling the pressure of breathable gas delivered to the patient by a flow generator;
receiving one or more signals from one or more sensors indicative of the pressure and/or flow of the gas in the system;
controlling the flow generator to deliver breathable gas at an exhalation pressure (EPAP) after delivering breathable gas at an inhalation pressure (IPAP) mode for a defined duration;
detecting inspiration based on when flow rate data indicates that the flow rate is above a trigger threshold and an exhalation check indicator indicates that the patient has previously exhaled; and
in response to detection of inspiration, delivering breathable gas in the IPAP.

12. The method of claim 11, wherein the defined duration is a machine breath duration.

13. The method of claim 12, wherein after the machine breath duration, the controller transitions to EPAP regardless of the flow rate.

14. The method of claim 11, wherein the defined duration is a maximum duration at the IPAP.

15. The method of claim 11 further comprising detecting exhalation based at least in part on whether the flow rate data indicates that the flow rate is below the cycling threshold.

16. The method of claim 15, wherein the trigger threshold and the cycling threshold have different flow rate threshold values.

17. The method of claim 11 further comprising, on transition to the IPAP, changing the expiration check indicator to indicate that exhalation has not occurred.

18. The method of claim 11, wherein the expiration check indicator is a Boolean operator

19. The method of claim 11 further comprising delivering breathable gas in the IPAP mode if inspiration is not detected within a backup time period.

20. The method of claim 11, wherein the mode trigger threshold varies during the inspiratory and/or expiratory cycles of breathing.
